(19) United States
(12) Patent Application Publication
(10) Pub. No.: US 2006/0246430 A1
(43) Pub. Date: Nov. 2, 2006

(54) Method and kit for the diagnosis or treatment control of intestinal carcinoma

(75) Inventors: Winfried Albert, Weilheim (DE); Pia Steffens, Hannover (DE); Alf-Andreas Krehan, Anzing (DE); Monica Stefan, Hannover (DE); Stefanie Waschuetza, Hannover (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: AdnaGen AG, hannover-Langenhagen (DE)

(21) Appl. No.: 10/488,728

(22) PCT Filed: Sep. 6, 2002
(86) PCT No.: PCT/EP02/09983

§ 371 Date: Nov. 22, 2004

(30) Foreign Application Priority Data
Sep. 6, 2001 (DE) 101437757

(51) Int. Cl. C12Q 1/68; C12P 19/34

(52) U.S. Cl. 435/6; 435/91.2

(57) ABSTRACT

The invention relates to a diagnosis kit and to a method for the diagnosis or treatment control of intestinal carcinoma in a human being. According to the invention, the presence or absence of at least two different messenger-RNAs coding for several of the tumor marker proteins CK20, EGF-R, CEA, GA733.2, PDGF-β and/or stanniocalcin is detected in a human blood sample. The presence of intestinal tumor cells in the blood sample is then deduced therefrom, as is possible metastatic spread.

The present invention concerns a procedure and a kit for diagnosis or monitoring of intestinal cancer in humans.

In cancer care, it is relevant to be able to detect a relapsing malignant tumor early by means of the appearance of metastasizing tumor cells in the blood. In the current study methods, so-called “tumor markers” at the protein level (immunological or enzymatic) are determined quantitatively in the blood or other body fluids of cancer patients.

These detection procedures are suitable for tumor diagnosis or monitoring/after-care only under certain conditions, since elevated tumor marker values can also be caused by nontumor diseases (e.g. inflammations of the gastrointestinal tract, cirrhosis of the liver, viral infections), excessive smoking, or by a pregnancy.

Tumor progression after resection of a primary colorectal tumor is primarily due to residual tumor cells. These cells are released from the primary tumor before or during the operation and have the possibility of spreading throughout the body.

In addition to the first recognition of a colorectal carcinoma, the earliest possible detection of metastasizing cells is therefore of critical importance for successful treatment. Likewise, in clinical stage I, a definitive negative finding can be helpful when it must be decided whether the patient must be treated with chemotherapy or an operation.

The currently used diagnostic methods are imprecise when evaluation of the malignant potency of residual tumors after chemotherapy has been performed in the metastasizing stage. Some clinical studies indicate a prognostic importance for disseminated tumor cells. Nevertheless, numerous methodological aspects are critical and have not yet been standardized sufficiently. Thus evidence of a hidden or residual metastasis must still be found that permit timely classification of primary curative therapeutic options.

Efforts to improve chances for healing have led today, on the one hand, to a search for and use of new tumor markers and, on the other, to improved sensitivity in the methods used.

The task of the present invention is to make available a procedure and a kit with which diagnosis or monitoring of intestinal cancer is possible in simple, certain, and repeatable manner.

This task is solved by the procedure according to claim 1 as well as the kit according to claim 33 and the microarray according to claim 56. Advantageous developments of the procedure, the kit and the microarray will be given in the various dependent Claims.

According to the invention, the presence or absence of mRNA from at least two of the tumor marker proteins CK20, EGF-R, DEA, GA733.2, PDGF-β, and/or stanniocalcin is detected in a blood sample of a human.

Since the RNAs of the markers described are normally not expressed in the blood of healthy persons, there is a direct correlation between positive RT-PCR evidence of these tumor markers and circulating tumor cells in the blood that can lead to metastasis.

Since individual markers can be expressed in different ways, depending on the therapy, it is advantageous to study a combination of two, three, four or more tumor markers, in order to detect all tumor cells circulating in the blood. In this way, tumor cells can be recognized even if the expression of a particular marker in a patient or in a stage of the disease is relatively low, which otherwise could lead to a false negative result. The use of additional markers, however, mostly encounters limits when mononuclear blood cells have a background expression (“illegitimate transcription”) that prevents an exact analysis.

For the recognition of intestinal tumor cells, therefore, according to the invention, one of the following combinations of markers, with which the problem mentioned can nevertheless be avoided, is recommended as especially advantageous:

CK20, EGF-R, CEA, and stanniocalcin;

CK20, EGF-R, GA733.2, CEA, and stanniocalcin;

EGF-R, CEA, and GA733.2;

GA733.2, CEA, and EGF-R;

GA733.2, CK20, and EGF-R;

GA733.2, PDGF-β, and EGF-R; or

GA733.2, PDGF-β, CEA, and EGF-R;

at least two markers selected from

CK20, EGF-R, and CEA; or

at least two markers selected from

EGF-R, CEA, and GA733.2.

The corresponding markers will be explained in the following.

| | | Alternative |
|---|---|---|
| Gene or gene product | Gene | name |
| Human carninoma-associated antigen GA733-2 gene | GA733-2 | GA733.2 |
| Human epidermal growth factor receptor (EGFR) gene | EGFR | EGFR |
| Human carcinoembryonic antigen (CEA) gene | CEA | CEA |
| Homo sapiens gene for cytokeratin 20 | CK20 | CK20 |
| Homo sapiens stanniocalcin 1 (STC1) gene | Stanniocalcin | Stanniocalcin |
| | 1 (STC1) | |
| Platelet derived growth factor-β | PDGF-β | PDGF-β |

In using the RT-PCR systems to detect tumor cells, specificity is a critical point because of the very high amplification rate. The slightest contaminations, such as from foreign RNA or illegitimate transcription, can make the results false in this case.

Through the use of immunocytochemistry with monoclonal antibodies against tumor cell antigens, an increase in specificity can be achieved by enriching the tumor cells with respect to the blood cells and simultaneously increasing the sensitivity of the tumor cell detection (detection rate of 1 tumor cell in 10⁷ mononuclear blood cells). In this case, the tumor cells are separated from mononuclear blood cells by means of specific antibodies or antibody mixtures. They can be separated by means of magnetic particles (Dynal) to which the antibodies are bound. This will be described in detail in the following.

Eukaryotic cells carry a number of different molecules on their cell surface. Depending on the origin and the function of the individual cells, the combination of the expressed surface molecules differs, so that cell type-specific patterns are formed. Antibodies are used to recognize these cell type-specific patterns. Antibodies bind with high specificity to their antigens, to selected surface molecules in this case. This characteristic is used to recognize cells and distinguish them from one another by means of specific antibody binding, by means of their cell type-specific patterns.

The expression of special surface proteins distinguishes tumor cells from untransformed cells of this cell type. Since this special pattern of surface antigens in tumor cells differs from the typical patterns of blood cells, tumor cells in the blood can be distinguished. In order to identify tumor cells, antibodies that specifically recognize these special surface proteins are used as tools. The specific antibody binding can be utilized for various analysis and separation methods.

Because of the intensive binding of immunoglobulins specially selected for this, it is also possible, in addition to recognizing cells by the surface epitopes, also to separate the recognized cells from those that have not been recognized.

1. Separation Principle Based on Liquid Phases; e.g. Flow Cytometry:

For flow cytometric analysis, antibodies are coupled with fluorescent dyes. Isolated cells are directed in a constant liquid flow individually to a light source (laser). When the cells are illuminated, the fluorescent dye bound to the antibodies is stimulated and radiates light of a particular wavelength. The radiated light is detected, and the signal measured is stored digitally. The light signal can be assigned to individual cells. The antibody-labeled cells are recognized in this way and can now be separated from other cells. For the separation, the cells are isolated in the smallest drops. After antibody-labeled cells have been recognized, the corresponding drops arrive at a collection container.

2. Separation Principle Based on Solid Phase; e.g. Magnetic Separation:

For magnetic separation, antibodies are coupled to pseudomagnetic particles. After the pseudomagnetic particles are placed in a magnetic field, the particles migrate in the magnetic field. During the movement in this magnetic field, cells to which these coupled antibodies are bound are carried along and separated from other cells.

For tumor cell recognition by means of magnetic particles, antibodies are consequently coupled covalently to pseudomagnetic particles that have a definite number of chemically activated sites on their surface. The specificity of the separation is determined by the specificity of the antibodies. A blood sample containing tumor cells is moved with antibody-coupled magnetic particles; then particles and blood are moved with respect to each other. The tumor cells that are recognized by the bound solid-phase antibodies and are thereby firmly attached follow the motion of the particles. In this way, it is possible, by applying a magnetic field, to pull the particles with the cells bound to them out of the blood (e.g. to the wall of the separation vessel). The blood that has been depleted of tumor cells can be replaced by other solutions, in which case the cells separated by the magnetic particles stay in place until the magnetic field is turned off/removed, and are available for further uses.

For recognizing tumor cells, specific antibody mixtures are used advantageously that are optimized either for tumor cells in general or else specifically for intestinal tumor cells. For example, a combination of antibodies MOC-31 and Ber-EP4 is suitable for recognizing tumor cells in blood.

Antibody mixtures of this kind show an increased sensitivity in comparison to antibodies used separately in cell recognition and cell separation, independent of the method used.

In the following, some examples of detecting intestinal tumor cells in blood samples according to the invention will be described.

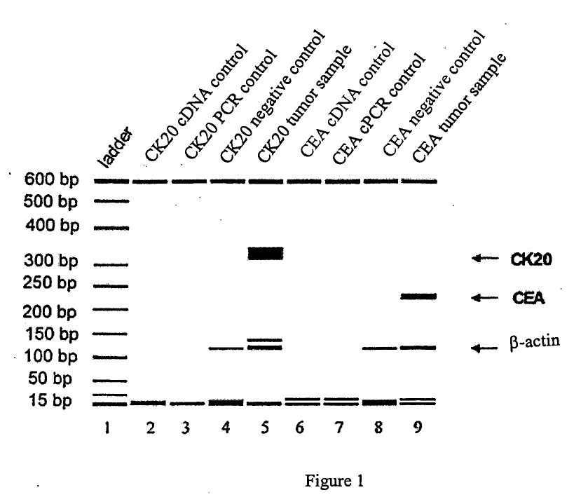

FIG. 1 shows detection of PCR products by electrophoresis;

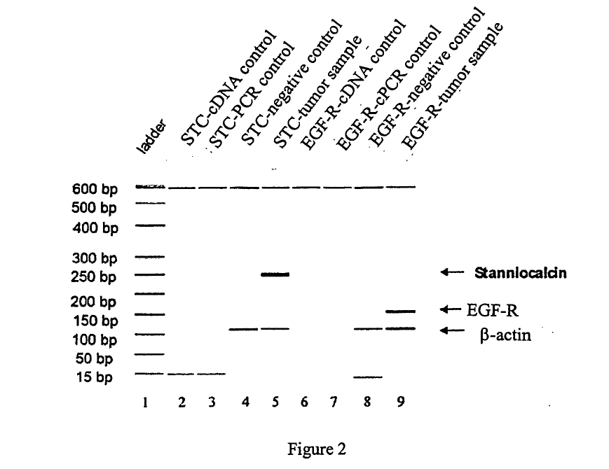

FIG. 2 shows another detection of PCR products by electrophoresis;

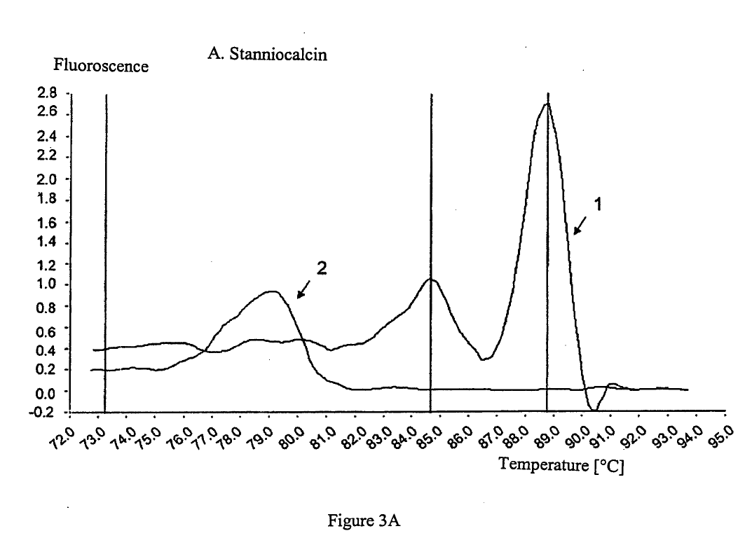

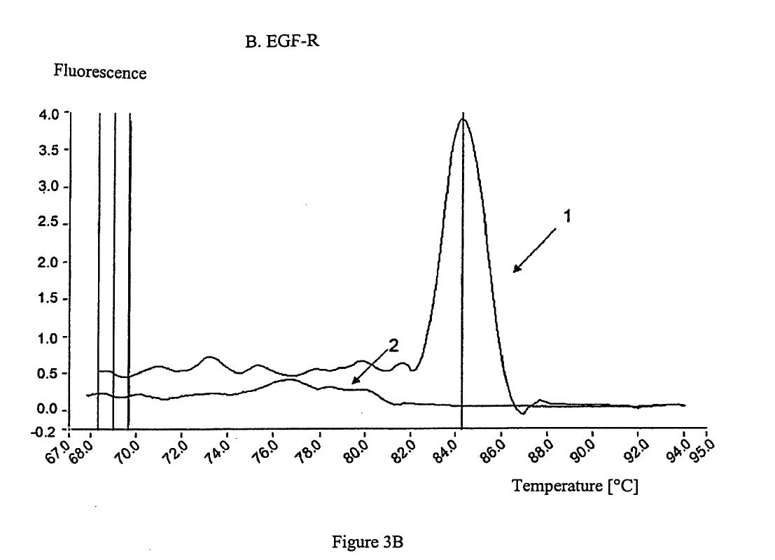

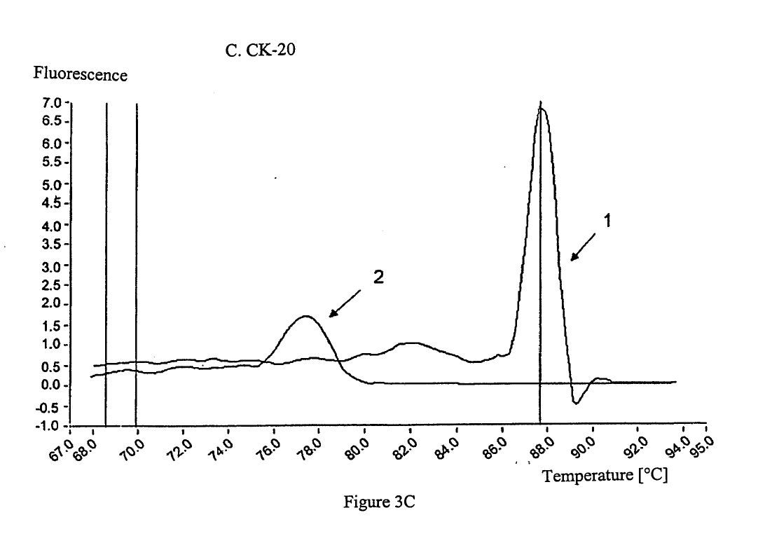

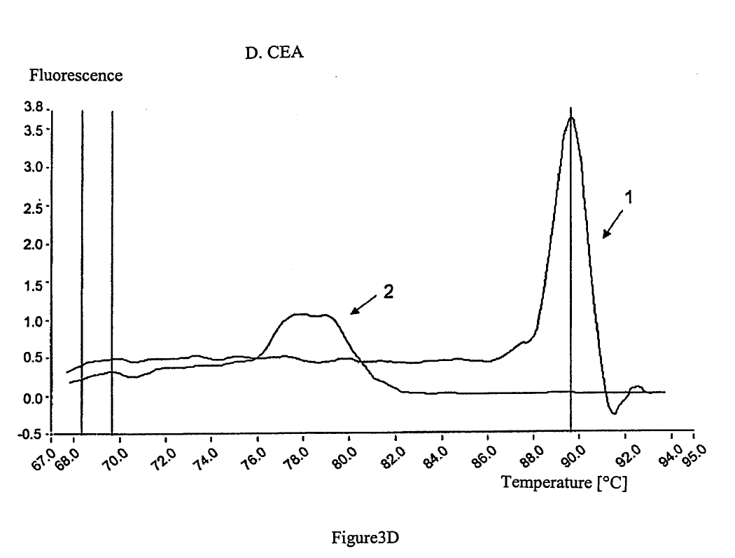

FIGS. 3A through 3D show tumor marker recognition by means of a LightCycler;

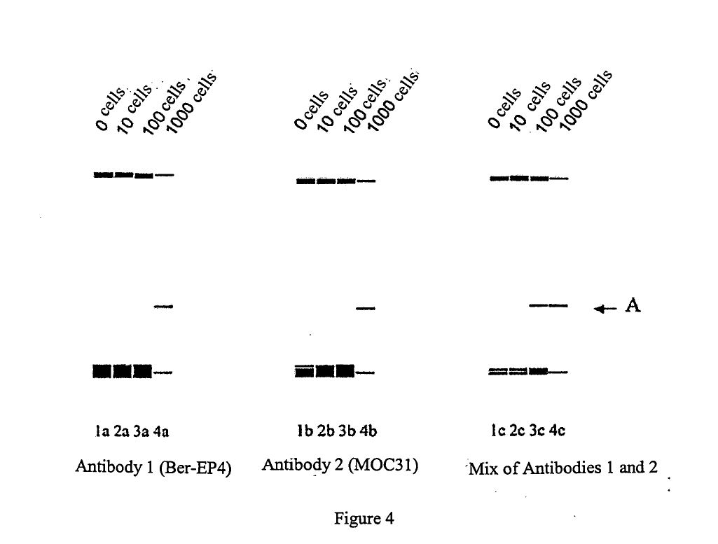

FIG. 4 shows detection of cell separation by means of antibody-labeled magnetic particles, and

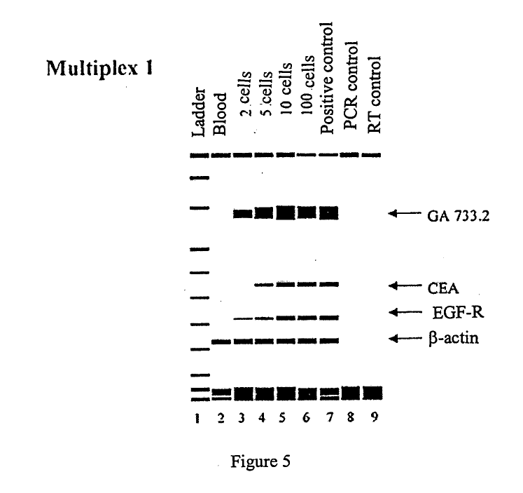

FIGS. 5 through 7 show the detection of intestinal tumor cells by various marker combinations.

In a first example, an RNA procedureing took place from 1 mL of EDTA whole blood with the QIAamp RNA Blood Mini Kit (Qiagen, Hilden). Contaminations with genomic DNA were avoided by an additional DNA digestion on the column by means of an RNase-free DNase Set), Qiagen, Hilden).

The RNA procedureing of 1 mL of EDTA whole blood was verified by photometry by the 260:280 nm ratios. For qualitative and quantitative determination in this case, 1 μL of the batch was analyzed by electrophoretic separation on an RNA 6000 chip with the Agilent Bioanalyzer 2100.

The isolated RNA was denaturized in an appropriate volume together with oligo(dT) 15 primers (Promega, Mannheim) for 5 min at 65° C. and then incubated directly on ice. The cDNA synthesis took place by means of the Sensiscript™ Reverse Transcriptase Kit (Qiagen, Hilden) in a 20-μL reaction batch according to Table 1 at 37° C. for 1 h, with subsequent inactivation of the reverse transcriptase for 5 min at 95° C., followed by cooling on ice.

TABLE 1
Components of cDNA synthesis

| Components | Volumes | | Final concentration |
|---|---|---|---|
| RNA | X | μL | 5 ng/μL |
| 10x RT buffer | 2 | μL | 1x |
| dNTP-Mix (5 mM each) | 2 | μL | 0.5 mM each |
| olig(dT) primer (10 μM) | 2 | μL | 1 μM |
| RNase inhibitor | 1 | μL | 0.5 units/μL |
| Reverse transcriptase | 1 | μL | 4 U |
| RNase-free water | to 20 | μL | |

With the cDNA generated, a multiplex PCR was performed for each of the selected tumor markers: Stanniocalcin, EGF-R, CK20, CEA, and, as an internal control, for β-actin. The PCR batch is presented in Table 2 below.

TABLE 2
PCR batch

| Components | Volumes | | Final concentration |
|---|---|---|---|
| cDNA | 8 | μL | |
| 10x PCR buffer* | 5 | μL | 1x |
| dNTP mix | 1 | μL | 200 mM each |
| Primer | (see Table 3) | | |
| DMSO additive*** | 1.0 | μL | |
| Taq DNA polymerase** | 0.5 | μL | 2.5 U |
| H₂O | to 20 | μL | |

(*contains 15 mM MgCl₂; for stanniocalcin 200 mM MgCl₂)
**HotStarTaq ™ DNA polymerase; Qiagen, Hilden
***DMSO additive with stanniocalcin

For each tumor marker, a primer pair was used, which can be seen in Table 3 below.

TABLE 3
List of PCR primers

| | | |
|---|---|---|
| | | PCR- |
| Primer name | Sequence 5′ → 3′ | Product |

Tumor marker

| | | |
|---|---|---|
| Stanniocalcin | AACCCATGAGGCGGAGCAGAATGA | 254 bp |
| sense | | |
| Stanniocalcin | CGTTGGCGATGCATTTTAAGCTCT | |
| antisense | | |
| EGF-R sense | AGTCGGGCTCTGGAGGAAAAGAAA | 163 bp |
| EGF-R antisense | GATCATAATTCCTCTGCACATAGG | |
| CK20 sense | ATCTCCAAGGCCTGAATAAGGTCT | 336 bp |
| CK20 antisense | CCTCAGTTCCTTTTAATTCTTCAGT | |
| CEA sense | AGAAATGACGCAAGAGCCTATGTA | 231 bp |
| CEA antisense | AACTTGTGTGTGTTGCTGCGGTAT | |
| GA733.2 sense | AATCGTCAATGCCAGTGTACTTCA | 395 bp |
| GA733.2 antisense | TAACGCGTTGTGATCTCCTTCTGA | |
| PDGF-β sense | TCTCTCTGCTGCTACCTGCGTCTG | |
| PDGF-β antisense | GTTGGCGTTGGTGCGGTCTATGAG | |
| Internal control | | |
| β-Actin sense | CTGGAGAAGAGCTACGAGCTGCCT | 116 bp |
| β-Actin | ACAGGACTCCATGCCCAGGAAGGA | |
| antisense | | |

The primer combinations and quantities used for detecting the individual tumor markers are listed in Table 4 below.

TABLE 4
Listing of primer quantities and primer combinations

| | Marker | | | |
|---|---|---|---|---|
| Primer | Stanniocalcin | EGF-R | CK20 | CEA |
| Stanniocalcin | 25 pmol | | | |
| sense | | | | |
| Stanniocalcin | 25 pmol | | | |
| antisense | | | | |
| EGF-R sense | | 25 pmol | | |
| EGF-R antisense | | 25 pmol | | |
| CK20 sense | | | 25 pmol | |
| CK20 antisense | | | 25 pmol | |
| CEA sense | | | | 25 pmol |
| CEA antisense | | | | 25 pmol |
| β-Actin sense | 1 pmol | 1 pmol | 1 pmol | 1 pmol |
| β-Actin antisense | 1 pmol | 1 pmol | 1 pmol | 1 pmol |

The PCR was performed under the PCR conditions given in Table 5 and with the marker-specific melting temperatures and number of cycles listed in Table 6.

TABLE 5
PCR conditions

| | | | | | |
|---|---|---|---|---|---|
| Preliminary | | 95° | C. | 15 min | |
| Denaturing | | | | | |
| Cycle | | | | | |
| | 1. Denaturing | 94° | C. | 1 min | |
| | 2. Annealing | x° | C. | 1 min | (see Table 6) |
| | 3. Extension | 72° | C. | 1 min | |
| Final | | 72° | C. | 10 min | |
| Extension | | | | | |
| | | 4° | C. | Pause | |

TABLE 6
Marker-specific annealing temperatures and number of cycles

Marker

| | | | |
|---|---|---|---|
| Stanniocalcin | EGF-R | CK20 | CEA |

Annealing temperature

| | 58° C. | 64° C. | 58° C. | 60° C. |
|---|---|---|---|---|
| Rotation rate | 45 | 40 | 35 | 40 |

1 μL of the PCR product generated in this manner was separated in an Agilent Bioanalyzer 2100 on a DNA chip (500), and the separation result was documented electronically. The results are shown in FIGS. 1 and 2. In these diagrams, lane 1 in each case shows a 100-kb line and lanes 2-9 the results from the corresponding samples. As can be seen, lane 5 shows a PCR product for tumor marker CK20 and lane 9 a PCR product for tumor marker CEA, while all samples with biological material (lanes 4, 5, 8, and 9) contain PCR products for the internal control β-actin.

In FIG. 2, lanes 5 and 9 show PCR products of the marker stanniocalcin or EGF-R, while the samples containing biological material according to lanes 4, 5, 8, and 9 show PCR products of β-actin as an internal marker.

Lanes 2, 3, 6, and 7 contained no biological material, so that there were no corresponding PCR products appeared there, either. The so-called cDNA control is a batch entirely without cDNA, and the negative control is a batch with RNA from a healthy control person in FIG. 1 and in FIG. 2. CEA stands for carcinoembryonic antigen, CK20 for cytokeratin 20, STC for stanniocalcin, and EGF-R for epidermal growth factor receptor in FIGS. 1 and 2.

FIG. 3 shows the alternative analysis by means of fluorescence-based real-time PCR by means of intercalation of fluorescent dyes.

This tumor marker detection can also take place by means of a LightCycler (Roche, Basel) as an alternative to block PCR.

The reverse transcription of mRNA takes place as described above. Then the PCR is performed with the LightCycler DNA Master Sybr Green I kit (Roche, Basel) according to the manufacturer's instructions under conditions optimized for each tumor marker. The oligonucleotides given in Table 3 are used as primers. Table 7 and Table 8 show the batch for PCR or PCR conditions in the LightCycler.

TABLE 7
PCR batch, LightCycler

| | Tumor marker | | | |
|---|---|---|---|---|
| Components | Stanniocalcin | EGF-R | CK20 | CEA |
| cDNA | 3.0 μL | 3.0 μL | 3.0 μL | 3.0 μL |
| MgCl₂ | 2.0 mM | 2.0 mM | 2.0 mM | 2.0 mM |
| Primer | 0.5 μM each | 0.5 μM | 0.5 μM | 0.5 μM |
| | | each | each | each |
| LightCycler DNA | 2 μL | 2 μL | 2 μL | 2 μL |
| Master Sybr Green | | | | |
| DMSO | 1 μL | — | — | — |
| H₂O | to 20 μL | to 20 μL | to 20 μL | to 20 μL |

TABLE 8
PCR conditions, LightCycler

| | Stanniocalcin | EGF-R | CK20 | CEA |
|---|---|---|---|---|
| Denaturing | 95° C., 30 sec, | 95° C., 30 sec, | 95° C., 30 sec, | 95° C., 30 sec, |
| | 20° C./sec | 20° C./sec | 20° C./sec | 20° C./sec |
| Amplification | 95° C., 5 sec, | 95° C., 5 sec, | 95° C., 5 sec, | 95° C., 5 sec, |
| | 20° C./sec | 20° C./sec | 20° C./sec | 20° C./sec |
| | 67° C., 10 sec, | 60° C., 10 sec, | 58° C., 10 sec, | 60° C., 10 sec, |
| | 20° C./sec | 20° C./sec | 20° C./sec | 20° C./sec |
| | 73° C., 15 sec, | 73° C., 15 sec, | 73° C., 20 sec, | 73° C., 20 sec, |
| | 20° C./sec | 5° C./sec | 5° C./sec | 5° C./sec |
| Melting curve | 95° C., 0 sec, | 95° C., 0 sec, | 95° C., 0 sec, | 95° C., 0 sec, |
| | 20° C./sec | 20° C./sec | 20° C./sec | 20° C./sec |
| | 70° C., 20 sec, | 65° C., 20 sec, | 95° C., 15 sec, | 95° C., 15 sec, |
| | 20° C./sec | 20° C./sec | 20° C./sec | 20° C./sec |
| | 95° C., 0 sec, | 95° C., 0 sec, | 95° C., 0 sec, | 95° C., 0 sec, |
| | 20° C./sec | 1° C./sec | 0.1° C./sec | 0.1° C./sec |
| Cooling | 30° C., 30 sec, | 30° C., 30 sec, | 95° C., 30 sec, | 95° C., 30 sec, |
| | 20° C./sec | 20° C./sec | 20° C./sec | 20° C./sec |

The result of this PCR and evaluation by LightCycler technology is presented in FIGS. 3A through 3D.

In all of FIGS. 3A through 3D, the control curve is designated by 2, while the curves that were recorded for the samples are designated by 1.

In this analysis, the melting curves of the PCR products were analyzed by Sybr Green I detection. Each of the graphs of FIGS. 3A through 3D shows in this case the fluorescence measured as a function of the temperature. The fluorescence peaks appearing in the control batches are attributable to primer dimers.

FIG. 3A represents here the melting curve analysis of the Stanniocalcin-PCR product. The melting point of the main product is 88.7° C. and the melting point of the byproduct is 84.6° C. Fluorescence peaks of this kind cannot be recognized in the control sample.

FIG. 3B shows the melting curve analysis of the EGF-R PCR product with a melting point of 84.3° C.

FIG. 3C shows the melting point analysis of the CK20-PCR product, with a melting point of 87.6° C.

FIG. 3D shows the melting curve analysis of the CEA-PCR product, with a melting point of 89.7° C.

Traditional analysis methods such as agarose-gel electrophoresis can also be used as alternatives to the methods presented here, in which case, for example, 25 μL of the PCR products presented above are separated through a 2.5% agarose gel and the DNA bands are then dyed and made visible with ethidium bromide. The documentation can be made, for example, with the aid of the DUO Store System from Intas.

A fragment analysis by means of the ABI Prism 310 Genetic Analyzer (PE Applied Biosystems Co., Weiterstadt) can also be used for evaluation. For this, a PCR with fluorescence-labeled primers is performed and then, for example, 1 μL of each PCR product is used at a dilution of 1:50.

Detection by means of sequence-specific fluorescence-labeled hybridizing samples is possible as additional evidence, which permits the product development to be monitored after each PCR cycle. By means of special standards, a conclusion can be drawn as to the quantity of starting RNA.

Enrichment of the cell fractions of the blood samples used for this is central to the quality of RNA used as the basis for detection and the cDNA synthesized from it. Four different methods are available for this:

a) Enrichment by Repeated Centrifugation after Erythrocyte Analysis:

1 mL EDTA blood is lysed after addition of 5 volumes of erythrocyte lysis buffer (“QIAmp Book Kit,” Qiagen, Hilden) for 20 min on ice. After removal of the plasma/lysate from the pelletized cells and resuspension, another centrifugation took place at 3000×g for 20 min. After the residue was removed, the pelletized leukocyte fraction is available for RNA preparation.

B) Enrichment Through Density Gradients—Centrifugation:

By means of density gradients generated through centrifugation, cells of different average volume densities can be separated from one another. Mononuclear blood cells are separated by means of a Ficoll-Hypaque gradient (Pharmacia, Uppsala, Sweden) and then washed twice with PBS/1% FCS.

c) Enrichment of Tumor Cells by FACS Flow Cytometry:

The mononuclear cells from the fraction enriched under b) are incubated with fluorescence-labeled mononuclear antibodies to tumor-specific surface proteins. The labeled cells are washed twice with PBS and then resuspended: 10⁷ cells in 1 mL PBS. A FACS Vantage SE flow cytometer (Becton Dickinson) is used to isolate the tumor cells. Data capture, instrument control, and data evaluation are done with the CellQuest program. The sorted cells are transferred into a 1.5-mL reaction container (filled with 1 mL PBS). The RNA can then be isolated as described above.

Alternatively, the isolated fraction of mononuclear blood cells, isolated according to one of the above procedurees, can be homogenized in trizol reagent (Gibco BRL, NY, USA). After chloroform extraction, the aqueous phase containing RNA in isopropanol is precipitated at −80° C. After being washed twice in 80% ethanol, the pellet is dried in air and then resuspended in RNA-free water.

After this RNA isolation, the reverse transcription and mRNA detection then take place as described above.

d) Enrichment of Tumor Cells by Immunomagnetic Separation.

The expression of special surface proteins distinguishes tumor cells from untransformed cells of this cell type. Since this special pattern of surface antigens in tumor cells also differs from the typical blood cell patterns, tumor cells in the blood are distinguished. In order to identify tumor cells, antibodies that recognize these special surface proteins are used as a tool. The specific antibody binding can be utilized for the procedure according to the invention. Antibodies are coupled covalently to pseudomagnetic particles that have a definite number of chemically activated sites on their surface. The specificity of the separation is determined by the specificity of the antibodies. A blood sample containing tumor cells is moved with magnetic particles coupled to antibodies. Two different mixtures of antibodies are used as antibodies in different examples; then particles and blood are moved with respect to one another, for example by “over-end rotation” of samples in a closed container or by moving the particles on the bases of alternating magnetic fields. The (tumor) cells that are recognized by the bound solid-phase antibodies and that are thereby firmly attached follow the motion of the particles. In this way, it is possible, by applying a magnetic field, to pull the particles with the cells bound to them out of the blood (e.g. to the wall of the separation vessel). The blood that has been depleted of tumor cells can be replaced by other solutions, in which case the cells separated by the magnetic particles stay in place until the magnetic field is turned off/removed, and are available for further uses.

TABLE 9
Antibody mixture 1

| Antigen | Clone | | Concentration | |
|---|---|---|---|---|
| Epith. rel. antigen | MOC-31 | (Novocastra) | 1.25 | μL/10⁶ cells |
| Epithelial antigen | Ber-EP 4 | (DAKO) | 0.924 | μg/10⁶ cells |

By means of the antibody mixture in Table 9, however, tumor cells are captured quite generally with high specificity. This is based on the selective expression of the specific surface proteins which distinguish cancer cells from other cells.

By using the antibody mixture, an increased sensitivity can be demonstrated quite fundamentally in comparison to antibodies used separately in cell separation, regardless of the methods used. This is shown in FIG. 4, in which in subdiagram A it is used with magnetic particles coated with BER-EP4 antibodies, in subdiagram B with magnetic particles coated with MOC-31 antibodies, and in subdiagram C with a mixture of particles coated separately with antibodies.

For each of the antibodies or the antibody mixture, a total of four measurements were made, in which 0, 10, 100, or 1000 carcinoma cells were inoculated into 10 mL of blood. Lanes 1a through 41, 1b through 4b, and 1c through 4c then show the detection of RNA after RNA preparation and RT-PCR with tumor-specific primers as described above for samples with a volume of 1 μL each. FIG. 4 was determined in this case by means of electrophoretic separation in an Agilent™ Bioanalyzer 2100 according the manufacturer's instructions.

When magnetic particles labeled with only one antibody are used, as in FIGS. 4A and 4B, positive detection is possible only with a content of 1000 cells. When an antibody mixture is used, as in FIG. 4C, detection was made already with only 100 cells, thus it is 10 times more sensitive.

In this example, experimental results were shown that do not represent the maximum possible sensitivity, but an example to demonstrate the improved sensitivity that can be achieved with the procedure according to the invention.

FIGS. 5 through 7 show the detection of marker combinations, whereby the conditions for the three multiplex reactions are given in the following tables for FIGS. 5 though 7.

Multiplex 1 (FIG. 5)

Marker

| | EGFR | CEA | GA733.2 | Actin |
|---|---|---|---|---|
| Primer conc. | 750 nM | 750 nM | 500 nM | 200 nM |

Temperature: 58° C.
40 cycles

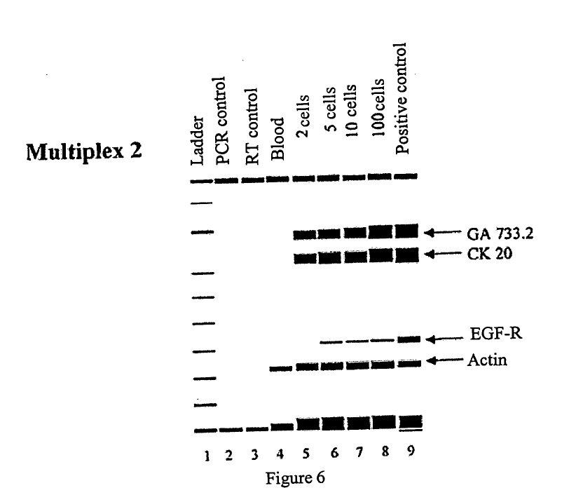

Multiplex 2 (FIG. 6)

Marker

| | EGFR | CK20 | GA733.2 | Actin |
|---|---|---|---|---|
| Primer conc. | 1 μM | 500 nM | 250 nM | 200 nM |

Temperature: 58° C.
35 cycles

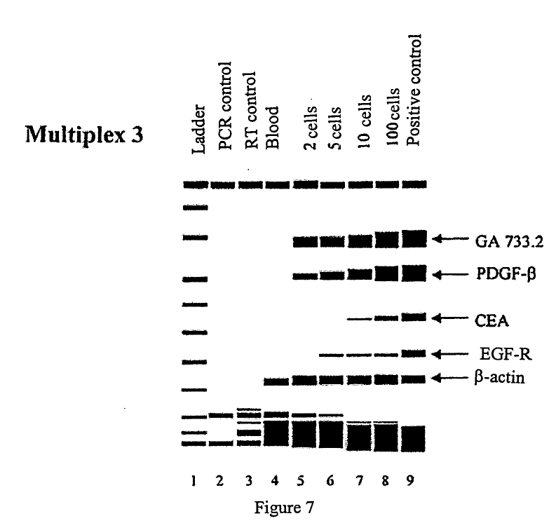

Multiplex 3 (FIG. 7)

| | Marker | | | | |
|---|---|---|---|---|---|
| | EGFR | CEA | PDGF-β | GA733.2 | Actin |
| Primer conc. | 1 μM | 1 μM | 500 nM | 250 nM | 200 nM |

Temperature: 58° C.
35 cycles

FIG. 5 shows the detection of intestinal cancer cells that have been inoculated into blood. Here, the selection of the cells was made with two antibodies, BER-EP4 and MOS-31. The molecular-biological detection step was performed with mRNA-markers GA733.2 (human carcinoma-associated antigen GA 733-2 gene), CEA (human carcinoembryonic antigen CEA gene), and EGF-R (human epidermal growth factor receptor gene). As can be recognized, detection could be made down to 2 cells in 5 mL of blood.

FIG. 6 shows a similar determination with tumor markers GA733.2, CK20 (Homo sapiens gene for cytokeratin 20), and EGF-R. As can be recognized, here too, sure detection was possible down to 2 inoculated intestinal tumor cells in 5 mL of blood.

FIG. 7 shoes detection by means of parallel determination of mRNA of markers GA733.2, PDGF-β (platelet-derived growth factor), CEA, and EGF-R. Here again, down to 2 inoculated cells per 5 mL of blood could be captured.

The diagnostic kit according to the invention and the procedure according to the invention also make it possible then to use the sorted and separated cells further as desired. This opens the possibility of studying the expression of other surface markers microscopically or also performing chromosome analyses. For this, the sorted cells are placed on object carriers. Other surface markers can be detected cytochemically or by fluorescence microscopy. Likewise, genetic analyses can be performed, such as chromosome analyses by means of FISH (fluorescence in situ hybridization) or karyogram compilation.

AACCCATGAGGCGGAGCAGAATGA
and
CGTTGGCGATGCATTTTAAGCTCT,
AGTCGGGCTCTGGAGGAAAAGAAA
and
GATCATAATTCCTCTGCACATAGG,
ATCTCCAAGGCCTGAATAAGGTCT
and
CCTCAGTTCCTTTTAATTCTTCAGT,
AGAAATGACGCAAGAGCCTATGTA
and
AACTTGTGTGTGTTGCTGCGGTAT,
AATCGTCAATGCCAGTGTACTTCA
and
TAACGCGTTGTGATCTCCTTCTGA,
and/or
TCTCTCTGCTGOTACCTGCGTCTG
and
GTTGGCGTTGGTGCGGTCTATGAG.

CTGGAGAAGAGCTACGAGCTGCCT
and
ACAGGACTCCATGCCCAGGAAGGA.

AACCCATGAGGCGGAGCAGAATGA
and
CGTTGGCGATGCATTTTAAGCTCT,
AGTCGGGCTCTGGAGGAAAAGAAA
and
GATCATAATTCCTCTGCACATAGG,
ATCTCCAAGGCCTGAATAAGGTCT
and
CCTCAGTTCCTTTTAATTCTTCAGT,
AGAAATGACGCAAGAGCCTATGTA
and
AACTTGTGTGTGTTGCTGCGGTAT,
AATCGTCAATGCCAGTGTACTTCA
and
TAACGCGTTGTGATCTCCTTCTGA,
and/or
TCTCTCTGCTGOTACCTGCGTCTG
and
GTTGGCGTTGGTGCGGTCTATGAG.

CTGGAGAAGAGCTACGAGCTGCCT
and
ACAGGACTCCATGCCCAGGAAGGA.

1. A procedure for diagnosis or monitoring of intestinal cancer in a human, which procedure comprises detecting in a blood sample from the human the presence of absence of at least two different mRNAs, wherein each of the at least two different mRNAs code for a tumor marker protein selected from the group consisting of CK20, EGF-R, DEA, GA733.2, PDGF-β and stanniocalcin, whereby detection of at least one of the mRNAs indicates the presence of intestinal tumor cells in the blood sample.

2. The procedure according to claim 1, which further comprises detecting the presence or absence of at least one mRNA that codes for a tumor marker protein selected from the group consisting of GA733.2, PDF-β, EGF-R and CEA.

3. The procedure according to claim 1, wherein tumor cells from the blood sample are separated or enriched, and the detection is carried out with the tumor cells.

4. The procedure according to claim 3, wherein the intestinal tumor cells are separated or enriched by means of antibodies generally specific for tumor cells, by means of antibodies specific for intestinal tumor cells, or mixtures of such antibodies.

5. The procedure according to claim 4, wherein the antibodies used for the separation of intestinal tumor cells have binding sites that bind to epitopes of an epithelial antigen and/or an epithelial membrane antigen.

6. The procedure according to claim 5, wherein the antibody is MOC-31, Ber-EP4, or a mixture thereof.

7. The procedure according to claim 1, wherein the intestinal tumor cells are separated or enriched by means of antibodies bound to magnetic particles.

8. The procedure according to claim 4, wherein the intestinal tumor cells are separated or enriched by means of fluorescence-associated flow cytometry, density gradient centrifugation, and/or centrifugation after erythrocyte lysis.

9. The procedure according to claim 8, wherein a centrifugation of blood samples is performed to pelletize the leukocytes contained in the blood.

10. The procedure according to claim 8, wherein the components of the sample that contain RNA are concentrated by means of lysing the erythrocytes contained in the sample and then pelletizing the leukocytes that are not lysed.

11. The procedure according to claim 8, wherein the components that contain RNA are concentrated by at least a density gradient centrifugation of the blood sample to separate and obtain the mononuclear blood cells contained in the sample.

12. The procedure according to claim 11, wherein the mononuclear blood cells obtained are labeled with fluorescence-labeled antibodies and separated and obtained from the sample by means of fluorescence-activated cell sorting (FACS).

13. The procedure according to claim 12, wherein the mononuclear cells of the fraction obtained are lysed and the mRNA is separated.

14. The procedure according to claim 1, wherein the RNA is isolated directly from the blood sample, and wherein the RNA is total RNA or mRNA.

15. The procedure according to claim 14, wherein following the isolation of the mRNA, a DNA digestion is performed.

16. The procedure according to claim 1, wherein the mRNA is reverse-transcribed into cDNA and the presence or absence of cDNA that codes for the tumor marker protein is detected.

17. The procedure according to claim 16, wherein at least one predefined segment of the cDNA is replicated by a polymerase chain reaction (“PCR”).

18. The procedure according to claim 17, wherein one or more oligonucleotide pairs that have the following sequences are used for replicating the cDNA:
AACCCATGAGGCGGAGCAGAATGA
and
CGTTGGCGATGCATTTTAAGCTCT,
AGTCGGGCTCTGGAGGAAAAGAAA
and
GATCATAATTCCTCTGCACATAGG,
ATCTCCAAGGCCTGAATAAGGTCT
and
CCTCAGTTCCTTTTAATTCTTCAGT,
AGAAATGACGCAAGAGCCTATGTA
and
AACTTGTGTGTGTTGCTGCGGTAT,
AATCGTCAATGCCAGTGTACTTCA
and
TAACGCGTTGTGATCTCCTTCTGA,
and/or
TCTCTCTGCTGOTACCTGCGTCTG
and
GTTGGCGTTGGTGCGGTCTATGAG.

19. The procedure according to claim 1, wherein the procedure further comprises detecting the mRNA that codes for of the protein β-actin as an internal control.

20. The procedure according to claim 19, wherein the mRNA that codes for β-actin is reverse-transcribed into cDNA and a segment of the cDNA is replicated by means of a polymerase chain reaction.

21. The procedure according to claim 20, wherein an oligonucleotide pair is used for replicating the cDNA that codes for β-actin,
whereby the oligonucleotides of the pair have the following sequences:
CTGGAGAAGAGCTACGAGCTGCCT
and
ACAGGACTCCATGCCCAGGAAGGA.

22. The procedure according to claim 17, wherein the replicated cDNA segment is digested using restriction enzymes, and the presence or absence of the mRNA that codes for a tumor marker protein is determined by means of the cDNA fragments produced.

23. The procedure according to claim 17, wherein a gel electrophoresis of the PCR products is performed to detect the amplified cDNA segments.

24. The procedure according to claim 17, wherein a fragment analysis is performed to detect the amplified cDNA segments.

25. The procedure according to claim 17, wherein during the course of the polymerase chain reaction, the fluorescence of the products generated is detected and the product development is detected.

26. The procedure according to claim 17, wherein the mRNA or cDNA is detected using a nucleotide microarray.

27. The procedure according to claim 26, wherein the PCR product is applied to a nucleotide microarray to detect the amplified cDNA.

28. A diagnostic kit for the diagnosis or monitoring of intestinal cancer, which kit comprises at least two pairs of oligonucleotides, whereby the two oligonucleotides of each pair are primers for amplification by means of a polymerase chain reaction of each of the two complementary strands of a desired DNA segment, and whereby the DNA segment amplified by each of the primer pairs comprises a portion of the cDNA that codes for a tumor marker protein selected from the group consisting of CK20, EGF-R, DEA, GA733.2, PDGF-β and stanniocalcin.

29. The diagnostic kit according to claim 28, wherein the kit contains at least three pairs of oligonucleotides, whereby the two oligonucleotides of each pair are primers for amplification by means of a polymerase chain reaction of each of the two complementary stands of a DNA segment, and
whereby each DNA segment comprises a portion of the cDNA that codes for a tumor marker protein selected from the group consisting of CK20, EGF-R, DEA, GA733.2, PDGF-β and stanniocalcin.

30. The diagnostic kit according to claim 28, wherein the kit contains an additional pair of oligonucleotides, which are primers for amplification of at least one segment of one of the two complementary strands of the cDNA that codes for the protein β-actin.

31. The diagnostic kit according to claim 28, wherein the two oligonucleotides of a pair have the following sequences, in a pair-wise manner:
AACCCATGAGGCGGAGCAGAATGA
and
CGTTGGCGATGCATTTTAAGCTCT,
AGTCGGGCTCTGGAGGAAAAGAAA
and
GATCATAATTCCTCTGCACATAGG,
ATCTCCAAGGCCTGAATAAGGTCT
and
CCTCAGTTCCTTTTAATTCTTCAGT,
AGAAATGACGCAAGAGCCTATGTA
and
AACTTGTGTGTGTTGCTGCGGTAT,
AATCGTCAATGCCAGTGTACTTCA
and
TAACGCGTTGTGATCTCCTTCTGA,
and/or
TCTCTCTGCTGOTACCTGCGTCTG
and
GTTGGCGTTGGTGCGGTCTATGAG.

32. The diagnostic kit according to claim 31, wherein at least one of the two oligonucleotides of a pair of oligonucleotides is labeled with fluorophores.

33. The diagnostic kit according to claim 32, wherein the oligonucleotides of different pairs are labeled with different fluorophores.

34. The diagnostic kit according to claim 30, wherein the kit contains a pair of oligonucleotides for amplification of the cDNA that codes for β-actin having with the following sequences:
CTGGAGAAGAGCTACGAGCTGCCT
and
ACAGGACTCCATGCCCAGGAAGGA.

35. The diagnostic kit according to claim 34, wherein at least one of the two oligonucleotides of the pair for amplification of the cDNA that codes for β-actin is labeled with fluorophores.

36. The diagnostic kit according to claim 28, wherein the kit contains the substances required for performing a polymerase chain reaction.

37. The diagnostic kit according to claim 36, wherein the kit contains a buffer solution, magnesium chloride, deoxynucleotide triphosphate and a heat-stable polymerase.

38. The diagnostic kit according to claim 37, wherein the heat-stable polymerase is a Thermus aquaticus polymerase (Taq polymerase).

39. The diagnostic kit according to claim 28, wherein the kit contains, as a positive control, at least one DNA sample that codes for a tumor marker protein selected from the group consisting of CK20, EGF-R, DEA, GA733.2, PDGF-β, and stanniocalcin.

40. The diagnostic kit according to claim 28, wherein the kit contains instructions for performing the polymerase chain reaction and/or instructions for performing a fragment analysis.

41. The diagnostic kit according to claim 28, wherein the kit contains a chart for evaluating the measurement results obtained.

42. The diagnostic kit according to claim 28, wherein the kit contains a microarray, whereby the array has a number of cells separated from one another, and an oligonucleotide is arranged in at least one cell of the microarray, which oligonucleotide hybridizes with the DNA segment.

43. The diagnostic kit according to claim 42, wherein in at least one additional cell of the microarray, another oligonucleotide is arranged and the sequence of the oligonucleotide arranged in the at least one additional cell differs from the sequence of the other oligonucleotides.

44. A microarray for diagnosis or monitoring of intestinal cancer, which comprises an arrangement of several cells separated from one another, wherein a different oligonucleotide is arranged in each cell, whereby each oligonucleotide hybridizes with a DNA segment that comprises a portion of the cDNA that codes for a tumor marker protein selected from the group consisting of EGF-R, CEA, and GA733.2.

45. The microarray according to claim 44, wherein one or more oligonucleotides are arranged in one or more additional cells, whereby each oligonucleotide hybridizes with a DNA segment that comprises a portion of the cDNA that codes for a tumor marker protein selected from the group consisting of CK20, PGDF-β and stanniocalcin.

46.-63. (canceled)


SEQUENCE LISTING (14 sequences)

SEQ ID NO: 1 - DNA, 24 bp, Artificial
  Oligonucleotide
aatcgtcaatgccagtgtacttca

SEQ ID NO: 2 - DNA, 24 bp, Artificial
  Oligonucleotide
taacgcgttgtgatctccttctga

SEQ ID NO: 3 - DNA, 24 bp, Artificial
  Oligonucleotide
agtcgggctctggaggaaaagaaa

SEQ ID NO: 4 - DNA, 24 bp, Artificial
  Oligonucleotide
gatcataattcctctgcacatagg

SEQ ID NO: 5 - DNA, 24 bp, Artificial
  Oligonucleotide
agaaatgacgcaagagcctatgta

SEQ ID NO: 6 - DNA, 24 bp, Artificial
  Oligonucleotide
aacttgtgtgtgttgctgcggtat

SEQ ID NO: 7 - DNA, 24 bp, Artificial
  Oligonucleotide
atctccaaggcctgaataaggtct

SEQ ID NO: 8 - DNA, 25 bp, Artificial
  Oligonucleotide
cctcagttccttttaattcttcagt

SEQ ID NO: 9 - DNA, 24 bp, Artificial
  Oligonucleotide
aacccatgaggcggagcagaatga

SEQ ID NO: 10 - DNA, 24 bp, Artificial
  Oligonucleotide
cgttggcgatgcattttaagctct

SEQ ID NO: 11 - DNA, 24 bp, Artificial
  Oligonucleotide
tctctctgctgctacctgcgtctg

SEQ ID NO: 12 - DNA, 24 bp, Artificial
  Oligonucleotide
gttggcgttggtgcggtctatgag

SEQ ID NO: 13 - DNA, 24 bp, Artificial
  Oligonucleotide
ctggagaagagctacgagctgcct

SEQ ID NO: 14 - DNA, 24 bp, Artificial
  Oligonucleotide
acaggactccatgcccaggaagga